(19) United States
(12) Patent Application Publication
(10) Pub. No.: US 2018/0078705 A1
(43) Pub. Date: Mar. 22, 2018

(54) THERMOPLASTIC TWO PIECE DISPOSABLE ASPIRATION SYRINGE

(71) Applicant: Joseph Alamat, Sterling Heights, MI (US)

(72) Inventor: Joseph Alamat, Sterling Heights, MI (US)

(21) Appl. No.: 15/272,507

(22) Filed: Sep. 22, 2016

(51) Int. Cl. A61M 5/31

(52) U.S. Cl. CPC A61M 5/3137; A61M 2005/3139; A61M 2005/3126

(57) ABSTRACT

A syringe for soft-tissue augmentation with a cosmetic filler material that facilitates one-handed aspiration and injection without changing grip position includes a one-piece plastic barrel having structure proximate a proximal end of the barrel for allowing a user to apply compression or traction between the barrel and a plunger assembly that is slidable in the barrel.

### FIELD OF THE DISCLOSURE

This disclosure relates to a syringe that can be used to facilitate injection of dermal filler or other material with the capacity to aspirate with the injection hand, without switching hands, prior to injection of material to avoid vascular injection.

### BACKGROUND OF THE DISCLOSURE

Non-surgical cosmetic rejuvenative procedures have become popular with patients because significant improvements can be achieved quickly, with minimal risks and recovery time. One technique that has become very popular involves soft tissue augmentation with a cosmetic filler material. Examples include gel-like compositions comprising silicone, hyaluronic acid, calcium hydroxylapatite, polymethylmethacrylate microspheres, and poly-L-lactic acid. Injection of these dermal fillers, typically subcutaneously, while generally safe and beneficial, is not completely without risk. One potential complication that can occur even with highly skilled and experienced practitioners is vascular injection and occlusion, which can lead to an embolus of the material traveling within the blood vessel and causing serious complication such as blindness. Vascular occlusion can occur when excessive external filler material exerts pressure on a blood vessel or by direct injection of the filler material into a vein or artery. Vascular occlusions can be avoided by aspiration prior to injection. If blood is aspirated, the needle needs to be repositioned. Current syringes require the insertion of the syringe with one hand, stabilization of the syringe with the other hand, grabbing the plunger with the first hand and pulling back to aspirate. If no blood is drawn, then the plunger is released and the syringe is reheld and the stabilizing hand is released and material injected. During the movement and exchange of hands, the needle tip will scrape against bone, or move in the tissue causing increased pain and trauma. The current design requires the syringe be inserted, the thumb pulls back and if no blood is drawn, then the material is injected. The needle tip will not move against bone or move in the tissue. This decreases trauma to tissue, pain and expedites the injection process.

This disclosure is directed to an improved disposable syringe that allows improved technique that reduces risk of complication during subcutaneous injection of soft tissue filler material.

This disclosure is also directed to an improved disposable syringe that allows improved technique that facilitates injection or aspiration of materials using one hand without changing grip or finger and thumb position between injection and aspiration.

### SUMMARY OF THE DISCLOSURE

Disclosed is a disposable plastic syringe that facilitates injection and aspiration with one hand without requiring a grip change.

The disclosed syringe includes structure that allows a plunger to move in either direction relative to the barrel using fingers and thumb, without requiring repositioning of the thumb or fingers.

In one embodiment, the syringe includes a one-piece plastic barrel having two finger loops at a proximal end of the barrel that allows insertion of fingers (e.g., the index and middle fingers), and a plunger assembly slidable in the barrel and having a thumb loop at a proximal end of the plunger assembly that allows insertion of a thumb. This arrangement allows a user to aspirate the syringe, without switching hands and prevents movement of the syringe in the tissues which causes pain and injury while switching hands. This determines if the tip of a hypodermic needle attached to the syringe is located in a blood vessel before injecting soft tissue filler material, thereby reducing risk associated with injecting filler material into a blood vessel.

In another embodiment, the barrel includes spaced apart ledges projecting from opposite sides of the barrel at a proximal end of the barrel, and a plunger assembly slidable in the barrel and having a thumb loop at a proximal end of the plunger assembly for insertion of a thumb. Fingers can be inserted into the gaps between the ledges to allow a user to apply compression or traction between the ledges and the thumb loop to inject or aspirate, respectively.

### BRIEF DESCRIPTION OF THE DRAWINGS

FIG. 1 is a perspective view of a syringe in accordance with this disclosure.

FIG. 1A is a plan view of a syringe in accordance with this disclosure.

FIG. 1B is a plan view of the syringe shown in FIG. 1A, with the plunger assembly separated from the syringe barrel.

FIG. 2 is a perspective of an alternative embodiment of a syringe in accordance with this disclosure.

### DETAILED DESCRIPTION

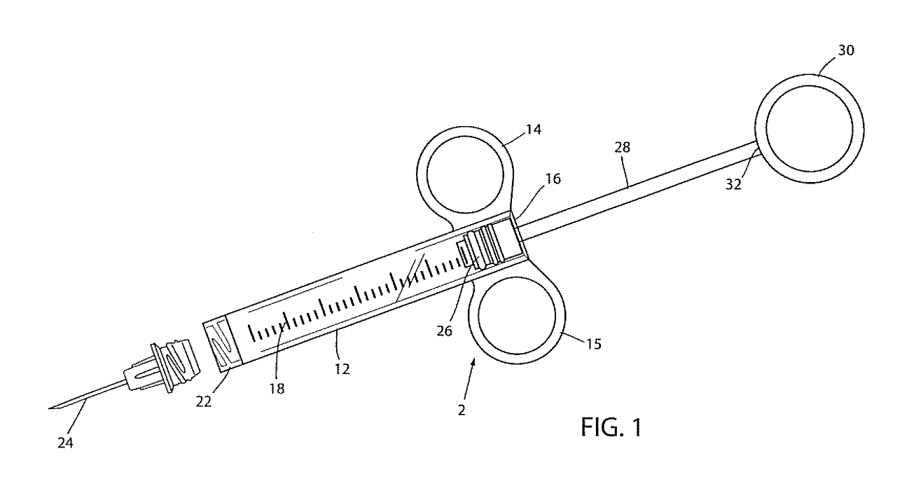

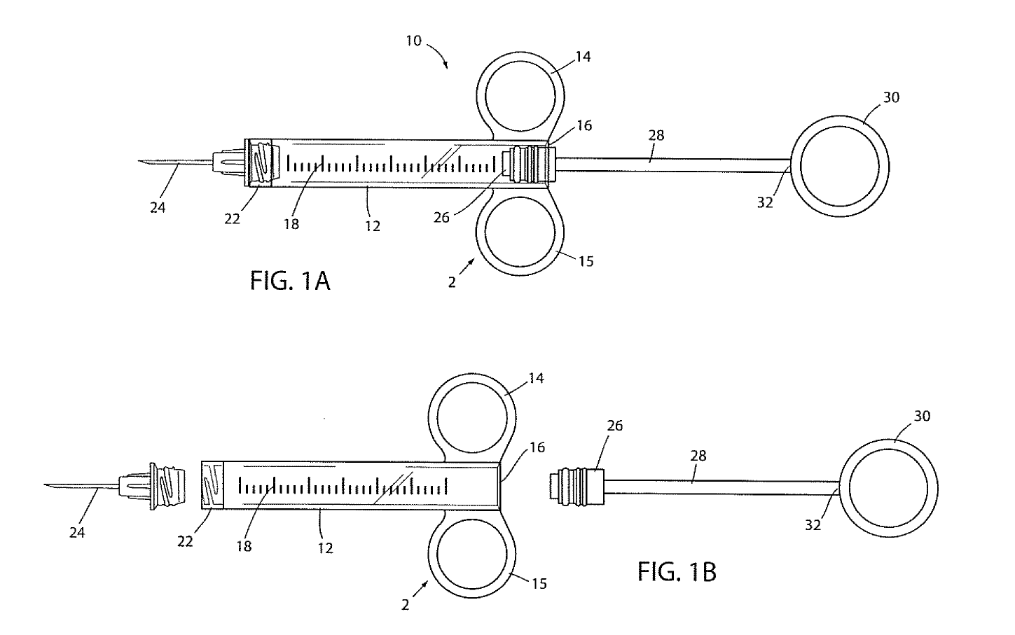

Shown in FIG. 1 is a syringe 10 comprising a one-piece plastic (thermoplastic or thermoset resin material) barrel 12 having two integral finger loops 14, 15 located at a proximal end 16 of barrel 12. Syringe 10 typically has a capacity of from about 0.2 cc to about 50 cc. In the illustrated embodiment, loops 14, 15 are circular or ring-shaped. However, loops having other shapes (e.g., square, octagonal, hexagonal, etc.) can be used. Loops 14, 15 can be made integral with barrel 12 by, for example, molding the loops and barrel together or by affixing the loops to the barrel, such as by using ultrasonic welding techniques or adhesives.

Barrel 12 is preferably transparent so that the contents of the syringe can be seen. Graduation markings 18 are preferably provided (e.g., printed or etched onto the outer surface of the barrel).

The distal end 20 of barrel 12 includes a luer lock or luer-slip style connector 22 for facilitating secure attachment of a hypodermic needle 24 to barrel 12.

Plunger assembly 26 is slidable along the inside of barrel 12 and includes a stem or shaft 28 having a thumb loop 30 at the proximal end 32 of the shaft 28. As with the finger loops 14, 15, illustrated thumb loop 30 is circular or ring-shaped, but could be formed in other shapes, such as square, hexagonal, octagonal, etc.

While loops 14, 15 of the illustrated embodiment are located at the proximal end of barrel 12, loops 14, 16 could be located somewhat distally of the proximal end. For example, loops 14, 16 could be located between the proximal end and the distal end, but nearer the proximal end than the distal end. Positioning of the loops on the barrel may depend on the capacity and length of the barrel and different configuration can be provided to accommodate personal preferences. Likewise different loop sizes and shapes can be provided to accommodate personal preferences.

Loops 14, 15 extend from opposite sides of barrel 12 (i.e., 180 degrees apart), but could project at different angles to accommodate personal preferences.

The plunger 26, shaft 28 and loop 30 can be made (e.g., molded) together from a thermoplastic or thermosetting material, or made separately and joined together (e.g., by ultrasonic welding or an adhesive).

The syringe can be prefilled with a soft tissue filler material, such as a composition comprising silicone, hyaluronic acid, calcium hydroxylapatite, polymethylmethacrylate particles or poly-L-lactic acid.

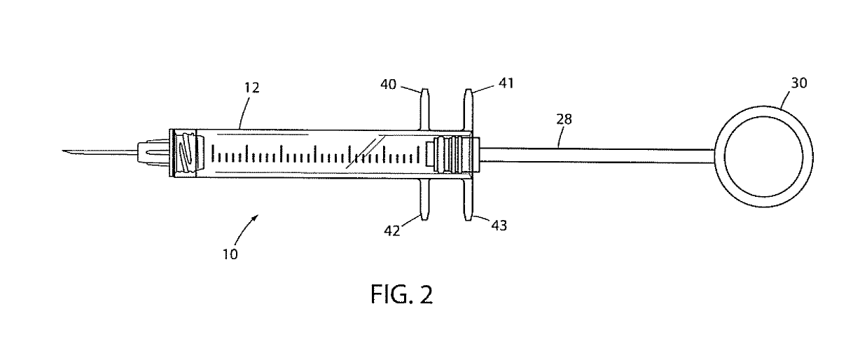

Shown in FIG. 2 is an alternative embodiment that is generally similar to the syringe shown in FIG. 1, but in which the finger loops 14, 15 are replaced with spaced apart ledges 40, 41 on one side of barrel 12 and spaced apart ledges 42, 43 on an opposite side of barrel 12. Fingers can be positioned between ledges 40, 41 and ledges 42, 43, and a thumb can be inserted through loop 30. Pressure can be applied to ledges 41, 43 and loop 30 to cause plunger assembly 26 to move distally to eject material from syringe 10, or traction can be applied via ledges 40, 42 to aspirate, without changing grip.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed is:

1. A syringe comprising:
A plastic barrel having two finger loops proximate a proximal end of the barrel that allow insertion of fingers; and
A plunger slidable in the barrel, the plunger having a thumb loop at a proximal end of the plunger that allows insertion of a thumb.

2. The syringe of claim 1, wherein the finger loops extend from opposite sides of the barrel.

3. The syringe of claim 1, wherein the barrel is transparent.

4. The syringe of claim 3, wherein the barrel has graduations to indicate the amount of injected material.

5. The syringe of claim 1, prefilled with a soft tissue filler material or any other material.

6. The syringe of claim 5, wherein the filler comprises a material selected from silicone, hyaluronic acid, calcium hydroxylapatite, polymethylmethacrylate and poly-L-lactic acid.

7. A syringe comprising:
A plastic barrel having spaced apart ledges projecting from opposite sides of the barrel proximate a proximal end of the barrel that allows insertion of fingers between the ledges; and
A plunger slidable in the barrel, the plunger having a thumb loop at a proximal end of the plunger that allows insertion of a thumb.

8. The syringe of claim 7, wherein the barrel is transparent.

9. The syringe of claim 8, wherein the barrel has graduations to indicate the amount of injected material.

10. The syringe of claim 7, prefilled with a soft tissue filler material.

11. The syringe of claim 10, wherein the filler comprises a material selected from silicone, hyaluronic acid, calcium hydroxylapatite, polymethylmethacrylate and poly-L-lactic acid.
